(19) United States
(12) Patent Application Publication
(10) Pub. No.: US 2022/0068016 A1
(43) Pub. Date: Mar. 3, 2022

(54) METHOD AND SYSTEM FOR DESIGNING ORTHOSES

(71) Applicant: FOOT CARE LTD, Rishon LeZion (IL)

(72) Inventor: Aviad RAZ, Rishon LeZion (IL)

(21) Appl. No.: 17/390,970

(22) Filed: Aug. 1, 2021

(30) Foreign Application Priority Data
Aug. 28, 2020 (IL) 276990

(51) Int. Cl. G06T 17/00; A61F 5/01

(52) U.S. Cl. CPC G06T 17/00; A61F 5/0127; G06T 2200/04; G06T 2219/012; G06T 2210/41

(57) ABSTRACT

A method and computer program product, the method comprising: receiving two or more images including a representation a foot of a patient, wherein the images are captured when the patient is lying with the patient's shin elevated, and wherein the patient is wearing a sock on the foot, the sock having attached thereto an object having known dimensions; generating a three-dimensional model of the foot from the images, comprising determining at least one dimension of the foot from a representation of the object in at least one of the at least two images; and creating a design of an orthotic in accordance with the three-dimensional model of the foot.

### CROSS REFERENCE TO RELATED APPLICATIONS

This application claims the benefit of priority of Israel Patent Application No. 276990, filed on Aug. 28, 2020. The foregoing patent is incorporated herein by reference in its entirety.

### TECHNICAL FIELD

The present disclosure relates to orthoses in general, and to a method and apparatus for designing and generating personally-adapted orthoses, in particular.

### BACKGROUND

An orthosis is an externally applied device used to modify the structural and functional characteristics of the neuromuscular and skeletal system. An orthosis may be used to: support, control, guide, limit and/or immobilize an extremity, joint or body segment for a particular reason. An orthosis may assist movement of a particular body part, for example by restricting the movement in a given direction, reducing weight bearing forces for a particular purpose, aiding rehabilitation from fractures after the removal of a cast, or otherwise correcting the shape and/or function of the body, to provide easier movement capability or reduce pain.

A lower-limb orthosis is an external device applied to a lower-body segment to improve function, by controlling motion, providing support through stabilizing gait, reducing pain through transferring load to another area, correcting flexible deformities, or preventing progression of fixed deformities. Lower-limb orthoses include various types of Ankle-foot orthoses, and foot orthoses.

Foot orthoses, also referred to as “orthotics” are devices inserted into shoes to provide support for the foot by redistributing ground reaction forces acting on the foot joints while standing, walking or running. Orthotics can have significant impact on foot, knee, hip, and spine deformities, and are aimed at aiding in a wide range of biomechanical deformities and a variety of soft tissue conditions, such as but not limited to painful high-arched feet, and may be effective for people with rheumatoid arthritis, plantar fasciitis, hallux valgus (“bunions”) or other problems.

Some orthotics are pre-molded (also referred to as pre-fabricated), while others are custom made, traditionally according to a cast or impression of the foot. If the orthotics fits well the user's foot, it can have a highly positive impact, while a poorly fitted orthotic can worsen the user's situation, cause pain and walking difficulties, or the like. Thus, it is important that the orthotics are well fitted to the user's foot.

### SUMMARY OF THE INVENTION

One exemplary embodiment of the disclosed subject matter is a computer-implemented method comprising: receiving two or more images including a representation a foot of a patient, wherein the images are captured when the patient is lying with the patient's shin elevated, and wherein the patient is wearing a sock on the foot, the sock having attached thereto an object having known dimensions; generating a three-dimensional model of the foot from the images, comprising determining one or more dimensions of the foot from a representation of the object in one or more of the images; and creating a design of an orthotic in accordance with the three-dimensional model of the foot. The method can further comprise enhancing the design by a user. Within the method, the object is optionally a coin. Within the method, the images are optionally frames from a video capture. Within the method, the patient is optionally lying face down. Within the method, the sock is optionally a patterned sock. Within the method, the images are optionally captured by a smart phone executing an application, wherein the application transmits the at least two images following said capturing. Within the method, the application is optionally adapted to receive verbal data related to the patient and transmit the verbal data in association with the images and wherein the verbal data is utilized in creating the design or enhancing the design. The method can further comprise receiving verbal data related to the patient, and wherein the verbal data is utilized in creating the design or enhancing the design. The method can further comprise: generating the orthotic in accordance with the design; and mailing the orthotics to the patient. Within the method, the object is optionally a reference shape made of non-stretchable material, and wherein the sock optionally further has marked or attached to its underside or bottom section a plurality of points or dots.

Another exemplary embodiment of the disclosed subject matter is a method comprising: receiving two or more images including a representation a foot of a patient, wherein the images are captured when the patient is lying with the patient's shin elevated, and wherein the patient is wearing a sock on the foot, the sock having attached thereto an object having known dimensions; generating a three-dimensional model of the foot from the images, comprising determining one or more dimensions of the foot from a representation of the object in one or more of the images; and creating a design of a pair of sandals or flip-flops in accordance with the three-dimensional model. The method can further comprise enhancing the design by a user. Within the method, the object is optionally a coin. Within the method, the images are optionally frames from a video capture. Within the method, the patient is optionally lying face down. Within the method, at least one of the socks is optionally a patterned sock. Within the method, the images are optionally captured by a smart phone executing an application, wherein the application transmits the at least two images following said capturing. Within the method, the application is optionally adapted to receive verbal data related to the patient and transmit the verbal data in association with the images and wherein the verbal data is utilized in creating the design or enhancing the design. The method can further comprise: generating the sandals or flip-flops in accordance with the design; and mailing the sandals or flip-flops to the patient. Within the method, the object is optionally a reference shape made of non-stretchable material, and wherein the sock optionally further has marked or attached to its underside or bottom section a plurality of points or dots.

Yet another exemplary embodiment of the disclosed subject matter is a sock for facilitating generation of the three dimensional mapping of a patient's foot, the sock comprising marked or attached to its underside or bottom section: a plurality of points or dots; and a reference shape made of non-stretchable material. Within the sock, the reference shape is marked or attached at a proximity of the center of the sole.

Yet another exemplary embodiment of the disclosed subject matter is a computer program product comprising a non-transitory computer readable medium retaining program instructions, which instructions when read by a processor, cause the processor to perform: receiving two or more images including a representation a foot of a patient, wherein the images are captured when the patient is lying with the patient's shin elevated, and wherein the patient is wearing a sock on the foot, the sock having attached thereto an object having known dimensions; generating a three-dimensional model of the foot from the images, comprising determining one or more dimensions of the foot from a representation of the object in one or more of the images; and creating a design of an orthotic in accordance with the three-dimensional model of the foot.

### THE BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1 shows a flowchart of steps in a method for designing and manufacturing an orthotic, in accordance with some exemplary embodiments of the disclosed subject matter;

FIG. 2 shows an illustration of a setup for capturing a patient's foot, in accordance with some exemplary embodiments of the disclosed subject matter;

FIG. 3 shows an illustration of determining a scaling factor for a foot model, in accordance with some exemplary embodiments of the disclosed subject matter;

FIG. 4 shows a user interface for designing an orthotic according to a model of a patient's foot, in accordance with some exemplary embodiments of the disclosed subject matter;

FIG. 5 shows a block diagram of an apparatus for generating personally fitted orthoses, in accordance with some exemplary embodiments of the disclosed subject matter;

FIG. 6A and FIG. 6B are side and bottom views, respectively, of a sock for facilitating generation of a three dimensional model of a patient's foot in a pre-worn state, in accordance with some exemplary embodiments of the disclosed subject matter; and

FIG. 7 is a bottom view of a sock for facilitating generation of a three dimensional model of a patient's foot in a worn state, in accordance with some exemplary embodiments of the disclosed subject matter.

### DETAILED DESCRIPTION

Orthotics, and in particular foot orthotics can significantly affect a patient's state, and specifically foot, knee, hip, and spine deformities, and can thus change for better or worse a patient's neuromuscular system while standing, walking, running or performing other activities.

In the description below, unless noted otherwise, the term patient refers to a person for whom orthotics are being designed and generated, or someone acting on his or her behalf. The term user refers to a professional such as a podiatric, or another person involved in the process of designing and generating the orthotics for the patient.

One technical problem of the subject matter is the need to design and generate personal orthotics for a patient, in accordance with the specific structure of the patient's foot, such that the orthotics will provide support where needed, flexibility where needed, adapt the height at different areas of the foot, or the like.

Another technical problem of the subject matter relates to the practical process of providing orthotics to a patient. It may be a significant burden for the patient, and in particular a patient living far away from a clinician, an old patient, or a patient with serious disabilities to arrive to the clinic, have the feet measured or imprinted, and then come back to pick the orthotics once ready.

Some known techniques can somewhat relieve this burden, by sending to the patient a stepping box containing a material into which the patient steps and which retains the foot imprint. The patient then sends back the box containing the foot print, the clinician generates the orthotics, and sends them to the patient. However, this process takes at least three mailing times in addition to the time it takes to generate the orthotics, incurs the associated cost of the box and the postage fees, and requires the production of the box and the imprint material which are needed only for a short time, after which they need to be disposed and create waste.

Other known techniques that somewhat improve the situation include the capturing of the patient's foot when the patient is sitting, such that the foot hangs down, or is placed on the floor. The foot may be captured using a digital camera or a video camera, and the photos or video are sent over a digital communication channel to the clinician, for example via e-mail, uploaded to a predetermined web site, or the like. The. The footage may then be to a computerized system that automatically determines the foot structure and designs corresponding orthotics, which may then be sent to the patient.

However, a computerized system receiving as input only the captured images cannot fully replace an experienced professional who has designed hundreds, thousands or more orthotics, and has learned from the experience gathered through interacting with the respective patients. Neither can the computerized system utilize verbal input from the patient describing the patient's needs, pains, limitations, or the like.

One technical solution comprises an orthotic designing system, that receives images or video captured taken when the foot is in a posture that is similar to the posture at walking, but without the forces applied to the foot by the stepping plane, such as the floor. Therefore, the images may be captured when the person is lying, with the shin raised, for example laid on a pillow, such that the foot is released. The user may be lying face down, such that in this posture the foot is at an angle similar to when the patient is walking, but without the patient's weight applied to it, such that the images capture the feet natural curvatures. The patient may wear a thin patterned sock, and have an object of a known size, such as a coin, attached to the sock, in order to make size calculations easier and more accurate. The object may be flat and may also be rigid. The object may be a part of the sock, for example built or stitched into the sock or attached to it in any manner, such as by Velcro®, adhesive tape, transparent adhesive tape, glued, stitched, or the like.

The images may be taken using a digital camera or a video camera, and transmitted over e-mail, uploaded or the like. Additionally or alternatively, the images may be captured using a smart phone executing an application which may comprise additional features, as detailed below. The transmission may comprise transmitting a video clip if one was captured, or two or more frames from the video.

The captured images or video may then be used for generating a three-dimensional model of the foot. Once the model is available, a corresponding orthotic may be designed.

The model generation or the orthotic design may be performed fully manually by a technician, a podiatrist, or the like, or by combining automatic and manual processes. For example, an initial model may be obtained by an automated system based on the images, followed by a process such as scaling or another fine tuning of the model. Similarly, an orthotic may be initially designed or selected by an automated process based on the foot model, followed by enhancement by a professional such as a podiatrist. The podiatrist may also take into account indications provided by the patient, whether when the images are captured, or at a later time, for example over the phone.

The designed orthotics may then be manufactured using any process such as molding, CNC, and sent by mail to the patient.

One technical effect of the disclosure provides for a method and system for providing a well fitted orthotic to a patient. The orthotic may be designed based on images of the foot in an advantageous posture, which is similar to a stepping posture, but does not suffer from the forces applied by the stepping plane. Thus, all the foot curvatures are clearly seen, which provides for a better fitting orthotic.

Another technical effect of the disclosure relates to the orthotics being generated and provided to the user in a fast manner, due to the usage of a computerized application, wherein only the final product needs to be physically mailed to the patient. The process also considers the environment by avoiding access trips of the patient or the podiatrist, and the creation and sending back and forth of auxiliary items such as a stepping box that later need to be disposed.

Yet another technical effect of the disclosure relates to utilizing the model of the foot for generating personally-fitted sandals or flip-flops for the patient. Such sandals or flip flops may comprise a flat or otherwise generally fitted sole, with internal face adapted in accordance with the user's feet model.

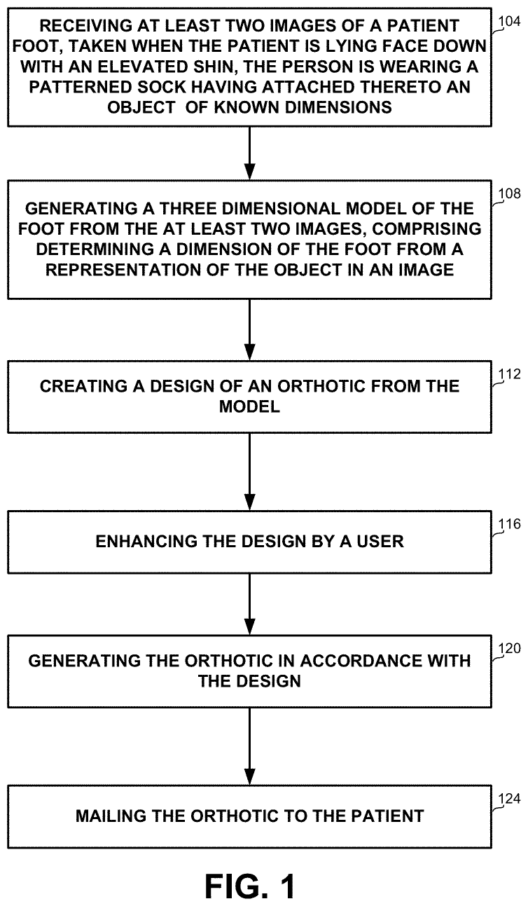

Referring now to FIG. 1, showing a flowchart diagram of a method for designing and manufacturing an orthotic, in accordance with some exemplary embodiments of the disclosed subject matter.

On step 104, one or more images of a feet for which an orthotic is required are obtained. The images may be captured when the patient is lying face down with the shin elevated, and the patient may be wearing a sock on the foot, the sock having attached thereto an object having known dimensions. The images may be captured as still images or as frames within a video clip. The images or video may be captured by a camera embedded within a smartphone, for example when executing an application. The sock may be patterned, wherein a patterned sock may be a sock having stripes of different colors, a checked sock, or a sock having a pattern with areas having distinct colors.

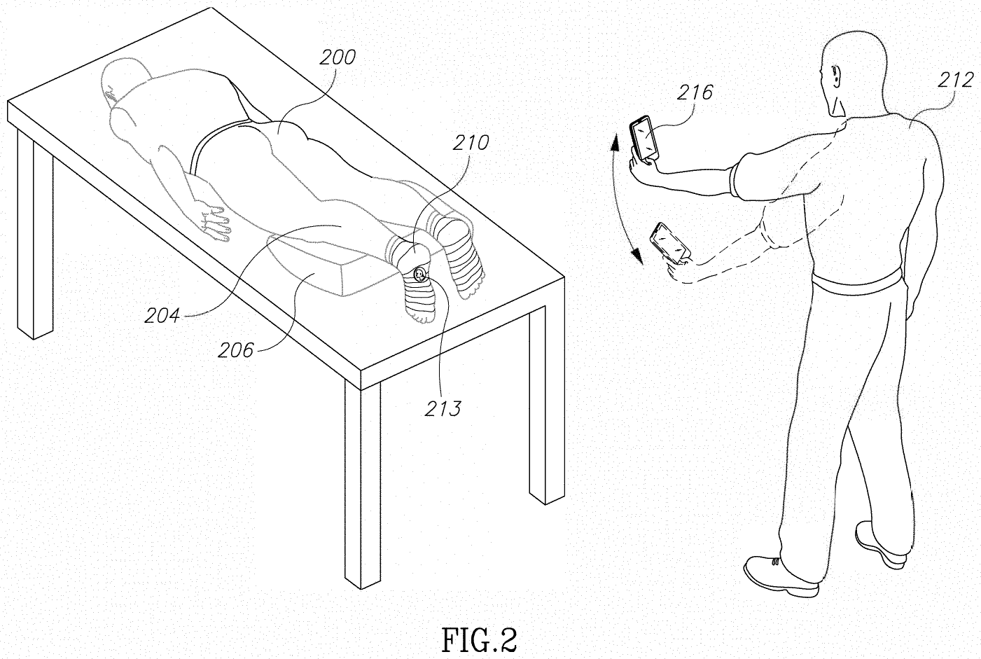

Referring now to FIG. 2, showing an illustration of a setup for capturing a patient's foot, in accordance with some exemplary embodiments of the disclosed subject matter.

After patient 200 or another person on his behalf has registered, the application may open the camera of smartphone 216 such that person 212 can start capturing the patient's foot.

Patient 200 is lying face down, with the patient's shin 204 being elevated, for example using a pillow 206 such that the patient's foot 208 is released. Patient 200 may be wearing a sock 210 which may be patterned, and may have a coin 213 of a known size, such as a quarter coin attached thereto, for example using a folded strip of adhesive tape, a double sided adhesive tape, a Velcro piece, or the like.

Another person 212 may use a smartphone 216 to capture the user's foot. Person 212 may take a few second video shot, for example 3-20 seconds or 5-10 second of the foot, from one end to the other, for example from heel to toes or from toes to heel. Person 212 may take an arch-like trajectory with the smartphone 216, such that all parts of the foot are captured at substantially equal distances.

During capturing, the application may monitor the lighting conditions and alert person 212 if the conditions are insufficient for the capturing. The application may also monitor the trajectory taken by the smartphone, such that if the trajectory is taken at a too high or too low speed, or is insufficiently arch-like, the application may notify person 212 such that person 212 may start over.

Additionally or alternatively, a pre-taken video or images may be uploaded to the application. The video or images may then be checked by the application as described above.

Additionally or alternatively, the application may request patient 200 or user 212 to watch the captured video, make sure the foot is seen well, and confirm it, before uploading the data to a predetermined web site.

The application may also enable patient 200, person 212 or another person to type or record verbal data, such as medical history of the patient, medical preferences, or the like.

The application may also enable the patient, user, to another person to provide additional data such as shoe size, images of existing shoes, preferred color, or the like.

Once done, the application may upload the images and all associated data, such as the verbal data and the preferences to a server, such as a server associated with the application.

However, it will be appreciated that while this embodiment comprises a dedicated application, in other embodiments, images or video may be captured by any capture device such as a digital camera or a video camera, and sent, for example using e-mail or an upload to a server.

On step 108, upon receiving the images, a three-dimensional model of the foot may be generated upon the video or images selected therefrom. It will be appreciated that the model is a data structure describing the foot, although a tangible model may also be created.

The model may be generated using any three-dimensional generation program, for example Agisoft Metashape® by Agisoft of St. Petersburg, Russia. The program may receive as input a number of images at one of a number of formats, such as but not limited to JPEG, JPEG 2000, TIFF, DNG, PNG, OpenEXR, BMP, TARGA, PPM, PGM, SEQ, ARA (thermal images) and JPEG Multi-Picture Format (MPO), or the like. The program may output a polygonal model, optionally with additional features such as color or texture associated with each polygon.

Such program may be used in one or more of a multiplicity of ways:

In one embodiment, the program can be accessed on a provider's servers, in the form of Software as a service (SaaS).

In another embodiment, the program may be installed on one or more computing platforms associated with the orthotic generating entity, and used as required. In some situations, if there are multiple orthotics to be generated, a processing queue may be implemented.

In a further embodiment, the program may be executed on a cloud computing platform such as Amazon Web Services (AWS), wherein payment may be in accordance with the actual usage time, number of activations, or the like.

As part of model generation step 108, or on a separate step, the model may be scaled. Scaling can be performed automatically using image analysis techniques, or by a user as detailed below.

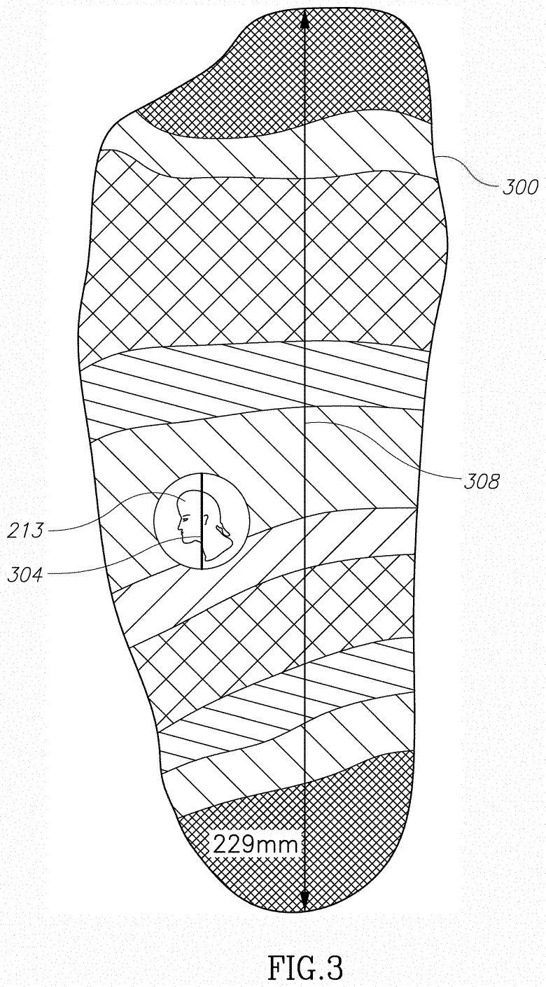

Referring now to FIG. 3, showing an illustration of determining a scaling factor for a foot model, in accordance with some exemplary embodiments of the disclosed subject matter. The model may be constructed upon a representation 300 of the foot in one or more images, and may be scaled in accordance with the representation of the object on the image. Coin 213, being a standard quarter coin, has a diameter of 0.955 inch or 24.26 mm. The size of the representation of coin 213 on the image, for example diameter 304, is measured, and the true dimensions of the foot may be calculated as shown by exemplary measure 308 by multiplying each imaged foot dimension by the ratio between the real size the coin and size 304 of its representation on the image. This yields a model that is scaled correctly to the true size of the foot.

On step 112, an orthotic may be designed in accordance with the model as scaled to size. In some embodiments, an initial orthotic design may be provided from a plurality of initial designs, may be sized in accordance with the dimensions of the patient's foot, and optionally enhanced to improve its fit to the user's foot. In some embodiments, the design may be performed in a single stage.

The stages above, e.g. selecting an initial design, sizing the design and enhancing the design, or providing a full design may be performed automatically, for example by dedicated software. In alternative embodiments, one or more of the stages may be performed by a user. For example, a user may select an initial design from a plurality of available designs, and enhance a final design generated automatically, for further improving the design, as detailed below.

A user may then continue to adapt the orthotic design to the needs of the patient's foot.

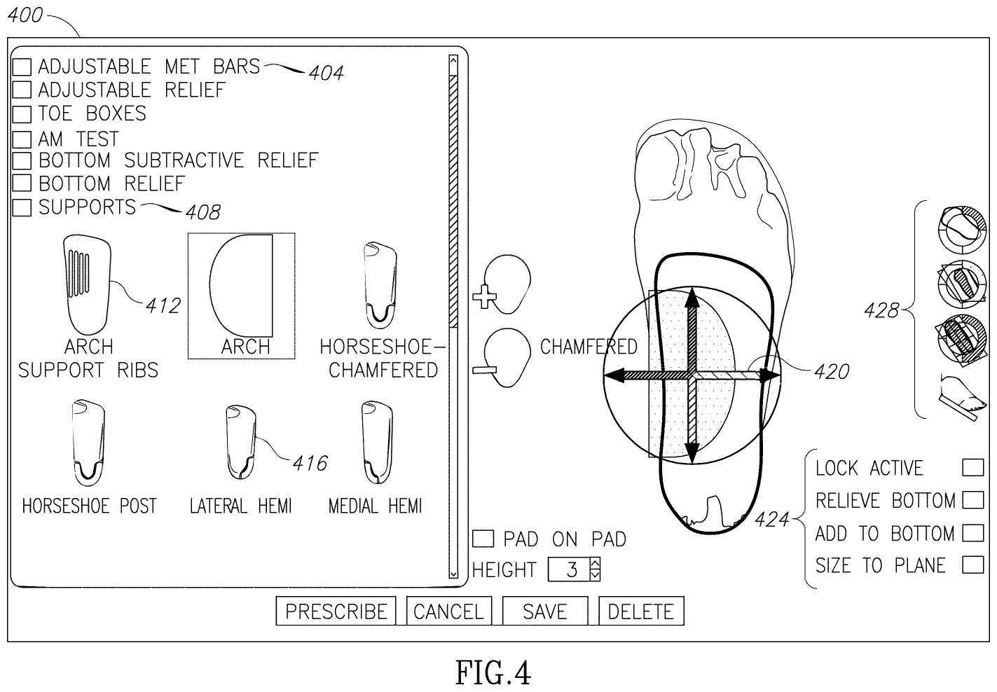

Referring now to FIG. 4, showing a user interface 400 to be used by a user such as a podiatric or an orthotic technician for designing an orthotic in accordance with a model of a patient's foot, in accordance with some exemplary embodiments of the disclosed subject matter. The orthotic 420 may be displayed, and the user may have a variety of selectable options for changing different aspects of the orthotic, for example adjustable met bars 404, supports 408 or others. Within each aspect, multiple options exist, for example in supports 408 the user can select arch support ribs 412, lateral hemi 416, or others.

The user can further adjust each such option, using a pointing device such as a mouse, a touch screen or the like, as shown by arrows 420.

Thus, a user may, for example, match the orthotics for the user's arch and/or the medial or lateral hemi, add height to the orthotic to compensate for one leg being shorter than the other, or the like.

It will be appreciated that the user interface may contain various other options or modes, such as options 424, modes 428 or the like. It will also be appreciated that the user interface shown in FIG. 4 is exemplary only, and may be designed in multiple other manners.

As part of the orthotic design, a user such as a podiatric, may read or listen to the verbal data provided by the patient or a person on his behalf using the application, or may speak with the patient, and enhance the orthotic based on the data.

On step 120, an orthotic may be generated in accordance with the design. It may be created in a multiplicity of ways, such as molding, computer numerical control machine (CNC), or the like.

In some embodiments, and with a slight variation, such as generating a sole and an upper, a pair of shoes, sandals or flip-flops may be generated for the patient upon the captured feet. The design may be selected by the patient, such that the end product is a personally fitted pair of sandals or flip flops.

On step 124 the finished orthotic may be mailed to the patient.

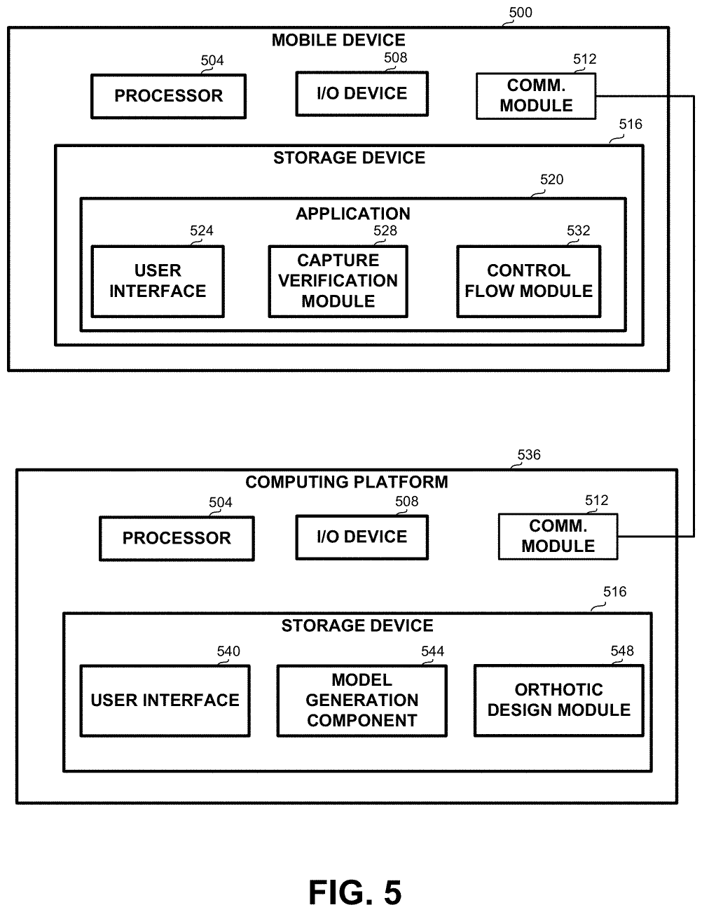

Referring now to FIG. 5, showing a block diagram of an apparatus for generating personally fitted orthoses, shoes, sandals or flip flops, in accordance with some exemplary embodiments of the disclosed subject matter.

The apparatus may comprise an application downloaded to and executed by a mobile device such as Smartphone 500, and a program executed by one or more Computing Platforms 536.

Smartphone 500 may comprise a Processor 504 which may be one or more Central Processing Units (CPU), a microprocessor, an electronic circuit, an Integrated Circuit (IC) or the like. Processor 504 may be configured to provide the required functionality, for example by loading to memory and activating the modules stored on Storage Device 516 detailed below.

Smartphone 500 may comprise Input/Output (I/O) Device 508 such as a camera, a display, a touch screen, a speakerphone, a headset, a pointing device, a keyboard, or the like. I/O Device 508 may be utilized to receive input from and provide output to a patient or another person, for example capture the patient's foot, walk the patient or another person through the process, or the like.

Smartphone 500 may comprise a communication module 512 for communicating with computing platform 536 via cellular communication, Wi-fi, Bluetooth, or the like.

Smartphone 500 may comprise a Storage Device 516, such as a hard disk drive, a Flash disk, a Random Access Memory (RAM), a memory chip, or the like. In some exemplary embodiments, Storage Device 516 may retain program code operative to cause Processor 504 to perform acts associated with any of the modules listed below, or steps of the methods of FIG. 1 above. The program code may comprise one or more executable units, such as functions, libraries, standalone programs or the like, adapted to execute instructions as detailed below.

Storage Device 516 may comprise Application 520, which may be downloaded to the Smartphone 500 and be used by the patient for registering and providing all the data required for fitting orthotics.

Application 520 may comprise User Interface 524, for displaying information to the patient or another person, and receiving input, for example obtaining from the user the registration details, providing instructions to the patient for the next steps to be taken, displaying to the patient the captured video and obtaining his confirmation, or the like.

Application 520 may comprise Capture Verification Module 528, for processing the captured video and verifying that it complies with the requirements, for example that the video is of a length between two predetermined thresholds, for example 3 seconds and 20 second, that the lightning conditions are adequate, that the trajectory of the smartphone relative to the foot is arch-like and not too angular, that the user's sock is patterned if requried, that the object such as the coin is visible, or the like.

Application 520 may comprise Control Flow Module 532 for controlling the process and the data flow, making sure all data is available at each stage and prompting the patient to take the next step, for example register with the system, capture the foot, view the video, provide textual or speech describing the relevant medical history or other data, and upload the materials using Communication Module 512.

Computing Platform 536 may comprise one or more computing platforms, for example one or more interconnected servers.

Computing Platform 536 may comprise a Processor 504, I/O Device 508, Communication Module 512 and Storage Device 516 as detailed above.

It will be appreciated that Processor 504 may be implemented as one or more processors, whether located on the same platform or not. It will also be appreciated that Computing Platform 536 may be implemented as one or more computing platforms which may be operatively connected to each other. For example, some components may be implemented as part of a first computing platform, while others may be implemented by a different computing platform providing FAAS, by cloud computing platform, or the like.

Storage Device 516 of Computing Platform 536 may comprise User Interface 440 for a user who may be a professional or another person performing acts related to designing or generating orthotics, as shown for example on FIG. 3 or FIG. 4. User Interface 540 may be operative in displaying the captured video to the user, displaying the foot model and the orthotics design to the user, and providing the user with tools for enhancing the model and the design, including measuring the size of the coin.

Storage Device 516 of Computing Platform 536 may comprise Model Generation Component 544 for generating a model of the foot based on the captured images. Model Generation Component 544 can be programmed to call a function or a service of another provider, for example a service provided by a third party.

Storage Device 516 of Computing Platform 536 may comprise Orthotic Design Module 548 for designing an orthotic based on the generated model. Orthotic Design Module 548 may use User Interface 540 for displaying to the user the initial design on the orthotic, which may be based on the model of the foot. The user can then make changes according to his or her professional expertise, using graphic tools such as modifying the arch, modifying the medial or lateral hemi, or the like.

When designing the orthotic, the user may also consider data provided by the patient that was provided by the patient or a person on his behalf, with the patient's foot images or on a conversation between the user and the patient.

In some embodiments, a personally adapted pair of sandals or flip-flops may be generated upon images of the two feet or a patient.

It will be appreciated that the module description above is exemplary only, that the modules may be arranged differently, and that the division of tasks between the modules may be different.

In some embodiments, a patient may be provided with a sock for facilitating the creation of a three dimensional model of the patient's foot. The sock may include, in accordance with some embodiments, multiple dots or spots of predetermined sizes optionally marked or positioned at predetermined distances from each other, over located on the entire foot sole, e.g. the underside or bottom section of the sock. The sole of the sock may further include a reference shape/bar of a known non-changeable and non-stretchable material, positioned substantially at, or at the proximity of, the center of the sole, for example within 10% of the center of the distance between the heel and the end of the first toe. The reference bar may be made of sturdy material such as plastic, wood, metal or others, and may be attached the sock using any required technology, such as but not limited to glued, stitched, or the like. The reference dots or spots may be made of the same material, but may also be made of flexible material which may change its size or shape when the sock is worn, such as silicone, rubber or the like.

According to some embodiments, upon being worn, the sole of the sock may stretch at or around certain areas of the foot, such as protruding, bulging or sticking-out areas, for example the heel, the ball and the fingers of the foot. The extent of stretching may correspond to the level of protruding, bulging or sticking-out. The sole of the sock may not stretch, or stretch to a lesser extent, at/around sunken/dented/flat areas of the foot of the wearing subject, for example the arch of the foot.

Thus, when one or more images of the sole of the worn sock are acquired as detailed above, the dots or spots on the sole of the sock may act as optical indicators/anchors and may be identified within the image(s). Distances between one or more identified dots or spots and their proximate/neighboring dots/spots may be compared to distances between other one or more identified dots or spots and their proximate/neighboring dots/spots to yield a relative dots or spots distance listing for the sole of the sock.

The disposition of the dots or spots over the sole of the sock may correspond to the levels of stretching of the different sections of the sole of the sock, wherein sole sections having a high disposition of dots or spots, i.e., relatively shorter distances between the dots or spots compared to other sole section are indicative of a non-stretched or less-stretched sock sole section, whereas sole sections having a low disposition of dots/spots, i.e., relatively longer distances between the dots or spots compared to other sole sections are indicative of a stretched sock sole section.

The reference shape or bar may be likewise identified within the image(s). Being of a known and substantially non-changeable and non-stretchable size, i.e., remains the same prior to and after wearing the sock, the size of the reference shape or bar may be used for calculating one or more of the distances in the relative dots or spots distances and/or the disposition map, to introduce values into the listed/shown distances and to calculate parameters of the foot.

Accordingly, the levels of stretching may be assessed by measuring the distances between dots or spots at different sections of the sole of the sock over an image capturing the foot, and may indicate the foot topography at their respective sections, collectively providing a three dimensional model of the foot, represented as a three-dimensional depth map, a three-dimensional point cloud or other types of a three-dimensional representation of the sole of the foot of the subject.

A three dimensional image reconstruction procedure of the patient's foot, in accordance with some embodiments, may include an image registration process wherein multiple images or multiple image frames—obtained from multiple viewpoints—of a video of the worn sock may be transformed into one coordinate system. Therefore, one or more of dots or spots, identified over multiple frames of the video, may be used as common or mutual reference points, allowing for the three dimensional alignment of the multiple video image frames and the generation of a three dimensional representation of the sole of the foot of the subject, based thereof.

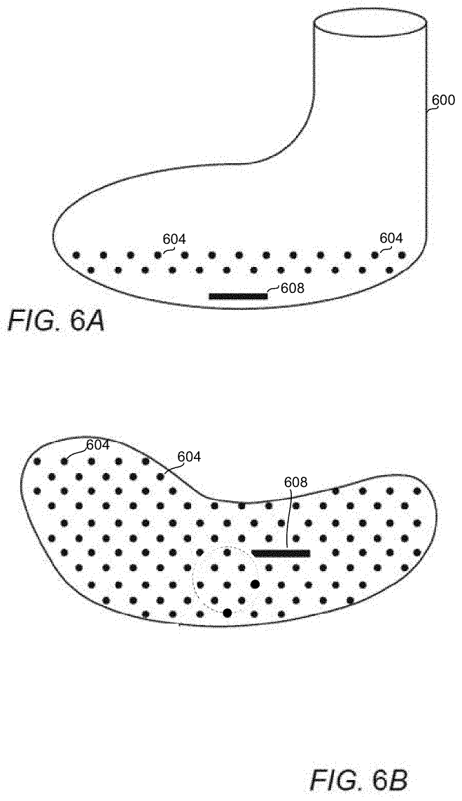

Referring now to FIG. 6A showing a side view and FIG. 6B showing a bottom view of an exemplary sock 600 for facilitating the generation of a three dimensional model of a patient's foot, wherein the sock is shown in its pre-worn form, in accordance with some embodiments of the disclosure. Sock 600 comprises a plurality of dots or spots 604. In the pre-worn form, dots or spots 604 are shown to be at similar distances from each other. Further shown in the figure is reference bar 608, made of sturdy material which would not stress when sock 600 is worn.

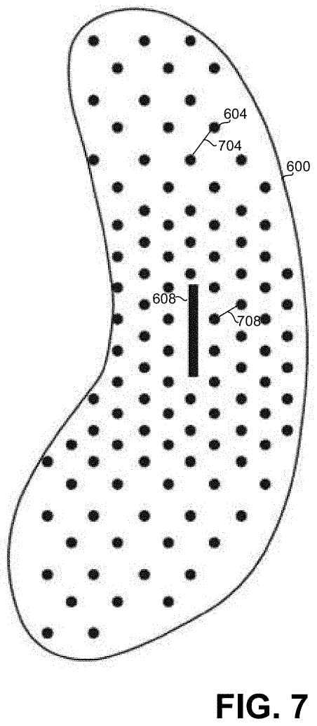

FIG. 7 shows a bottom view of sock 600 wherein the sock is shown in its worn form, in accordance with some embodiments of the present invention. In the worn form, at least some of dots or spots 604 are shown to be at various distances from each other. For example, distance 704 between dots or spots 604 at/around the heel or the ball of the foot, is larger than distance 608 between dots or spots 604 located around the mid-section or the foot arch section of the sole.

Thus, in some exemplary embodiments, an actual distance between dots at a particular area of the foot may be calculated by real size of reference bar (608)*imaged distance between dots (704 or 708)/imaged size of reference bar (608). Additionally or alternatively, the size of the dots in one or more dimensions may also be measured and used for assessing the stretching level at the dimension within an area and thus contribute to constructing the three dimensional model. Thus, a three dimensional model of the foot may be calculated using the reference bar real size and imaged size, and the distances between the dots and/or their dimensions as imaged.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method comprising:
receiving at least two images including a representation a foot of a patient, wherein
the at least two images are captured when the patient is lying face down with the patient's shin elevated, and wherein
the patient is wearing a sock on the foot, the sock having a part thereof, or an object attached thereto, having known dimensions;
generating a three-dimensional model of the foot from the images, comprising determining at least one dimension of the foot from a representation of the object in at least one of the at least two images; and
creating a design of an orthotic in accordance with the three-dimensional model of the foot.

2. The method of claim 1, further comprising enhancing the design by a user.

3. The method of claim 1, wherein the object is a coin.

4. The method of claim 1, wherein the at least two images are frames from a video capture.

5. The method of claim 1, wherein the at least two images are captured by a smart phone executing an application, wherein the application transmits the at least two images following said capturing.

6. The method of claim 6, wherein the application is adapted to receive verbal data related to the patient and to transmit the verbal data in association with the at least two images, and wherein the verbal data is utilized in creating the design or enhancing the design.

7. The method of claim 1, further comprising:
generating the orthotic in accordance with the design; and
mailing the orthotics to the patient.

8. The method of claim 1, wherein the object is a reference shape made of non-stretchable material, and wherein the sock further has marked or attached to its underside or bottom section a plurality of points or dots.

9. A method comprising:
receiving at least two images including a representation a feet of a patient, wherein
the at least two images are captured when the patient is lying face down with the patient's shin elevated, and wherein
the patient is wearing a sock on at least one of the feet, the sock having a part thereof, or an object attached thereto having known dimensions;
generating a three-dimensional model of the feet from the images, comprising determining at least one dimension of the feet from a representation of the object in at least one of the at least two images; and
creating a design of a pair of sandals or flip-flops in accordance with the three-dimensional model.

10. The method of claim 11, further comprising enhancing the design by a user.

11. The method of claim 11, wherein the object is a coin.

12. The method of claim 11, wherein the at least two images are frames from a video capture.

13. The method of claim 11, wherein the sock is a patterned sock.

14. The method of claim 11, wherein the at least two images are captured by a smart phone executing an application, wherein the application transmits the at least two images following said capturing.

15. The method of claim 14, wherein the application is adapted to receive verbal data related to the patient and transmits the verbal data in association with the at least two images, and wherein the verbal data is utilized in creating the design or enhancing the design.

16. The method of claim 11, further comprising:
generating the pair sandals or a flip-flops in accordance with the design; and
mailing the sandals or a flip-flops to the patient.

17. The method of claim 11, wherein the object is a reference shape made of non-stretchable material, and wherein the sock further has marked or attached to its underside or bottom section a plurality of points or dots.

18. The method of claim 11, wherein the sock comprises marked on or attached to its underside or bottom section:
a plurality of points or dots; and
a reference shape made of non-stretchable material.

19. The method of claim 18, wherein the reference shape is marked or attached at a center of a sole of the patient.

20. A computer program product comprising a computer readable storage medium retaining program instructions, which program instructions when read by a processor, cause the processor to perform a method comprising:
receiving at least two images including a representation a foot of a patient, wherein
the at least two images are captured when the patient is lying face down with the patient's shin elevated, and wherein
the patient is wearing a sock on the foot, the sock having a part thereof, or an object attached thereto, having known dimensions;
generating a three-dimensional model of the foot from the images, comprising determining at least one dimension of the foot from a representation of the object in at least one of the at least two images;
creating a design of an orthotic in accordance with the three-dimensional model of the foot.
